(19) United States
(12) Patent Application Publication
(10) Pub. No.: US 2026/0221265 A1
(43) Pub. Date: Jul. 30, 2026

(54) Foundation Model for Clinically Applicable Deformable Registration in Medical Imaging

(71) Applicant: Siemens Healthineers AG, Forchheim (DE)

(72) Inventors: Sureerat Reaungamornrat, Havertown, PA (US); Heyuan Huang, Austin, TX (US); Ioan-Marius Popdan, Brasov (RO); Ali Kamen, Skillman, NJ (US); Florin-Cristian Ghesu, Baiersdorf (DE)

(21) Appl. No.: 19/039,886

(22) Filed: Jan. 29, 2025

(51) Int. Cl. G16H 30/40; G06N 3/088; G06T 3/14

(52) U.S. Cl. CPC G16H 30/40; G06N 3/088; G06T 3/14

(57) ABSTRACT

For deformable registration in medical imaging, various approaches may be used alone or in combination to address the deficiencies of prior machine learning approaches, providing a machine learning approach that performs well and rapidly for clinical use. In one approach, the machine learning model is defined as multi-resolution with gradients in optimization passing between the resolutions. In another approach, the objective function used for unsupervised learning uses gradient information for similarity, consistency of direction between resolutions, and/or background penalties. In yet another approach, the spatial transforms between the physical coordinate system, the voxel grid indexing, and/or the machine learning coordinate system are used in application and/or training.

### BACKGROUND

The present embodiments relate to deformable registration between imaging data in medical imaging.

Registration has been performed using hand coded or programmed approaches and machine learning approaches. Programmed approaches exhibit long computation time due to their iterative nature, which, in turn, limits their applications in real clinical settings. Machine learning-based approaches (such as supervised, unsupervised, weakly-supervised learning) often involve a single iteration (i.e., single forward pass), making them more feasible for real-time usage. The ability of the existing machine learning approaches to model physically plausible deformation that respect tissue properties and motion patterns of tissue and organs is, however, inferior to programmed approaches. The machine learning approaches are less capable of modeling motion of detailed structures due to hardware memory limitations during training. Most deep learning (DL)-based approaches are not applicable to multimodality registration since they rely on single-modality image similarity metrics or metrics with a linear intensity relation assumption, such as mean squared difference (MSE) or normalized cross correlation (NCC). More advanced image similarity metrics, such as modality independent neighborhood descriptor-based (MIND) metrics, require large memory footprints, further accentuating the hardware limitations. Learning image similarity metrics in iterative approaches leads to the issue of long computation time.

Regardless of whether the machine learning approaches learn a spatial mapping or an image similarity metric, the machine learning approaches often perform poorly on unseen data. Inferior performance may be due to the inability of DL approaches to generalize. The inferior performance may be due to the insufficient number of model parameters and a small set of training data existing approaches used to train the models. These models perform well on test data whose distributions are close to the distribution of their corresponding training dataset but perform poorly on a test distribution that is far from their training set.

Another reason for the inapplicability of DL approaches in clinics is the unclear unit and coordinate system of the output deformations. The input images may be in a different coordinate system than the output deformations. Each coordinate frame has its own unit. Since the unit and the coordinate frame of the estimated deformation are unclear, mapping the estimated deformation back to the physical coordinate frame of the input images is nontrivial. Also, since the unit of motion is unclear, the spatial Jacobians of deformations computed by the existing DL approaches to impose smoothness do not necessarily induce smoothness or bijectivity.

### SUMMARY

By way of introduction, the preferred embodiments described below include methods, systems, computer programs, and non-transitory computer readable media for deformable registration in medical imaging. Various approaches may be used alone or in combination to address the deficiencies of prior machine learning approaches, providing a machine learning approach that performs well and rapidly for clinical use. In one approach, the machine learning model is defined as multi-resolution with gradients in optimization passing between the resolutions. In another approach, the objective function used for unsupervised learning uses gradient information for similarity, consistency of direction between resolutions, and/or background penalties. In yet another approach, the spatial transforms between the physical coordinate system, the voxel grid indexing, and/or the machine learning coordinate system are used in application and/or training.

In a first aspect, a method is provided for deformable registration by a medical imaging system. An image processor inputs first imaging data and second imaging data to a machine-learned model. The machine-learned model generates, in response to the input, a deformation field deforming the second imaging data to the first imaging data. The deformation field is in a coordinate system of the machine-learned model. The deformation field is spatially transformed from the coordinate system of the machine-learned model to a physical coordinate system. The second imaging data is deformed by the deformation field as transformed. A medical image of the patient from the second imaging data as deformed is displayed.

In a second aspect, a method is provided for machine training for deformable registration by a medical imaging system. A machine learning model is defined with input of multiple image data sets and output of a deformation field relating the multiple image data sets. A processor optimizes the machine learning model using an objective function. The objective function includes a factor for image background contribution penalty and/or for level consistency between different resolutions. The machine learning model as optimized is stored.

In a third aspect, a method is provided for machine training for deformable registration by a medical imaging system. A machine learning model is defined with input of multiple image data sets and output of a deformation field relating the multiple image data sets. A processor optimizes the machine learning model using an objective function. The objective function includes a first factor for similarity, the similarity measured with gradient information. The machine learning model as optimized is stored.

In a fourth aspect, a system is provided for image registration in medical imaging. An interface is configured to receive first and second images representing a patient, the first and second images from different imaging modalities. A memory is configured to store a machine-learned network. The machine-learned network comprises a multi-resolution network having been trained with gradients passing between resolutions of the multi-resolution network in optimization of the multi-resolution network. A processor is configured to input the first and second images to the machine-learned network, the machine-learned network configured to output a deformation field in response to the input. The interface is configured to output the deformation field.

Any one or more of the aspects, approaches, or concepts summarized above or in the Illustrative Embodiments below may be used alone or in combination. The aspects or concepts described for one Illustrative Embodiment or aspect may be used in other embodiments or aspects. The aspects or concepts described for a method or system may be used in others of a system, method, or non-transitory computer readable storage medium.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart of an embodiment of a method for machine training for deformable registration in medical imaging;

FIG. 2 illustrates an example multi-resolution machine learning model;

FIG. 3 illustrates the example model of FIG. 2 with example connections between resolution networks for gradients;

FIG. 4 is a block diagram showing one embodiment of a system for distributed training;

FIG. 5 illustrates different coordinate systems and corresponding transformations;

FIG. 6 is a flow chart of an embodiment of a method for clinical application of deformable registration in medical imaging using a machine-learned model; and

FIG. 7 is a block diagram of a system, according to one embodiment, for deformable registration in medical imaging.

### DETAILED DESCRIPTION OF THE DRAWINGS AND PRESENTLY PREFERRED EMBODIMENTS

DL-based deformable registration is not applicable in clinics due to (i) ignorance of image spatial information (i.e., image orientation, spacing, and origin) and/or (ii) an inferior ability to resolve deformation in unseen data. Existing DL-based registration algorithms do not incorporate spatial information necessary for fusion and integration of image signals in image guided intervention and therapy. In additional to disregarding spatial information, the unit of motions and their coordinate system estimated by existing DL approaches are unclear, making coordinate system conversion to the physical coordinate systems for clinical application impossible or difficult. The unclear unit and frame of motion, additionally, may lead to incorrect imposition of regularization/penalty in training, adversely affecting the model performance. The image spatial information is incorporated in training and/or application, making a DL-based deformable registration algorithm readily applicable in real clinical workflows. Due to the clear definition of the coordinate systems, correct regularization may be used in training, promoting physically plausible deformation in application.

In one approach, a coordinate system transformation is used in the DL-based registration algorithm. In another approach, a general network architecture promotes the learning of all network parameters through robust gradient flow and encourages the understanding of global and local features. In another approach, a distributed training scheme is used, allowing the maximum number of model parameters per available computing resource. In yet another approach, regularizations reduce the solution space and maximize the number of parameters per output dimension (here, output dimension is the multiplication of the spatial dimension and the number of voxels/pixels whose motions are to be computed, e.g., the output dimension of deformable registration of images whose size is 256×256×256=16,777,216 voxels is 3×16,777,216=50,331,648). As another approach, the regularization uses the lightweight multimodality image similarity metric.

The spatial transform approaches are readily applicable and integrate into existing clinical workflows. The spatial coordinate system transformation mapping between DL-library grid coordinate system, voxel index coordinate system, and physical coordinate system as defined in image metadata (in DICOM headers) allows the use of estimated deformations in any clinical applications without any modification to existing applications.

The correct imposition of priors promotes physically plausible motions. The spatial coordinate systems allow the clear definition of the unit of motions and the image coordinate system in which the estimated motions reside, which, in turn, allow correct computation and imposition of regularization/penalties based on spatial Jacobians. This avoids regularization in existing DL approaches that, instead of inducing smoothness, promote tearing and folding of tissues.

The main reasons for the inferior performance on unseen data are (i) the use of a small training dataset and (ii) the insufficient number of model parameters (‘model brain/memory’) to learn and understand complex organ interaction and a variety of locally varying motions. Although, for other applications, the design of neural network architectures does not have direct and severe impacts on the network performance. For 3D deformable registration, for a given available computing resource, hardware memory available to store the network parameters during training is limited (i.e., a large amount of memory space is required to hold intermediate 4D network features (3D spatial plus dimension for features), 3D inputs, and 3D output displacement fields). A network design that allows the model to learn global and local spatial features from images (benefiting its ability to resolve deformation) and to make use of all the parameters to learn necessary image features through robust gradient flow across model parameters and multiresolution levels is used. In addition, to allow a maximum number of model parameters per output dimension per available computing resources, a distributed training scheme that, besides partitioning data, divides, and trains model parameters in multiple machines simultaneously, may be used. This increases the network memory size which, in turn, improves the network's ability to learn a multitude of locally varying motion encountered in real clinical data and a variety of complex organ interaction.

A sufficient number of model parameters per output dimension is linked to model performance. Most prior work does not mention this since their tasks are not deformable registration (i.e., do not involve multiple 3D and 4D images/features) or their tasks are deformable registration trained and tested on a small set of data. Examples of tasks that are not deformable registration, so they had a sufficient number of parameters per output dimension include but are not limited to ResNet-50 for classification (23000 parameters per output dimension) and GPT-3 (400,000,000 parameters per output dimension). Due to 3D/4D features involved in deformable registration and limited computing resources, deformable registration, on the other hand, could undesirably have as small a number of parameters as 4 per output dimension, without proper design and distributed training.

The architecture design and/or distributed training result in a more effective network for deformable registration. Unlike other tasks where memory-to-stored network parameters is relatively abundant, for 3D deformable registration, most hardware memory has to be dedicated to storing inputs, outputs, and intermediate 4D features. A network design and a training scheme allow the network to contain as many as possible of parameters per available computing resources. The network design promotes learning of both global and local image structures/features and encourages all network parameters to capture necessary image feature information through robust gradient flows throughout the network and across multiresolution levels in training. In addition, to allowing the network to have as many parameters as possible per available computing unit, distributed training in which both model parameters and data are partitioned across computing units, addresses memory issues in training.

In addition to network design and the distributed training scheme, penalties are used to reduce the solution space (i.e., space of all possible deformations) to the space modeling physically plausible deformation and to enlarge the number of parameters per output dimension (i.e., increasing ‘model memory/neuron’ per output dimension). This improves the model's ability to learn and understand complex anatomical motions. Besides the limited number of parameters per output dimension, existing DL approaches demonstrate inferior performance on multimodality registration since they are based on single-modality image similarity metrics and/or metrics assuming a linear intensity relation. A lightweight (low computation cost) multi-modality image similarity metric promoting image alignment regardless of imaging modalities may improve performance.

The model is trained for deformable registration between images of the same imaging modality or different imaging modalities (multi-modality registration). For multi-modality registration, a lightweight (low computation cost) multimodality image similarity metric is used. The similarity metric is gradient information (GI). Distinct from MSE, GI does not assume the input images sharing the same intensity distribution. Unlike of NCC-based metrics, GI does not assume a linear relation between intensity distributions of the input images. MIND and mutual information (MI) are common metrics for modality registration, but the former requires a large memory footprint (inappropriate for DL-based 3D deformation registration whose DL model is already not large enough to navigate the large space of possible anatomy deformations) while the latter is computationally complex and sensitive to the sample size (i.e., local calculation is not reliable) and typical image degradation (i.e., intensity nonuniformity, artifacts).

By reducing the solution space, the network may have more parameters. To help the network learn within the large space of possible deformations with the limited number of parameters due to limited computing resources, one or more of various penalties may be used to constrain or reduce the solution space to the space modeling physically plausible anatomical deformations. This allows for an increase in the number of parameters per output dimension (i.e., increase ‘model innate memory/knowledge’ for each output dimension). Besides the typical penalties, such as diffusive smoothing penalty, bending energy, and/or inverse consistency, a multiresolution-level consistency constraint is used to enlarge the number of parameters per output dimension while, at the same, reducing the solution space. A background penalty may reduce the solution space to the space where deformation outside body is as close to zero as possible.

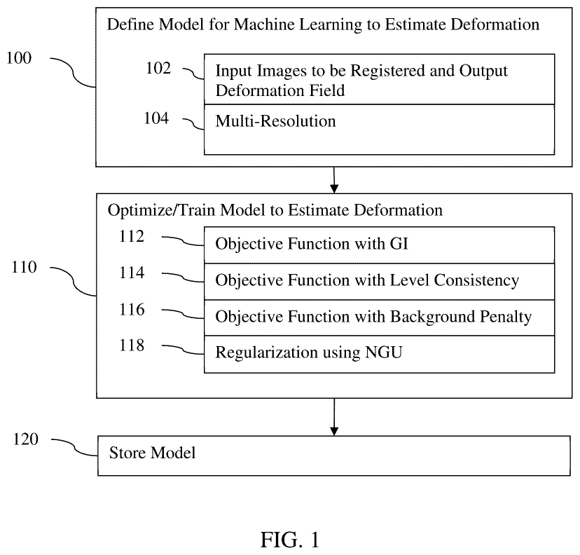

FIG. 1 shows a method for machine training for deformable registration by a medical imaging system. A model is trained to generate a deformation field given input imaging data from a same or different modalities. For example, imaging data for ultrasound and imaging data for computed tomography (CT) or magnetic resonance (MR) are to be registered. As another example, imaging data for CT is to be registered with MR. In yet another example, pre-operative imaging data is to be registered with intra-operative or post-operative imaging data. As another example, MR from one pulse sequence (e.g., T1-weighted) is to be registered with MR from another pulse sequence (e.g., proton density weighted). 2D data is registered with 2D data, or 3D data is registered with 3D data.

The method of FIG. 1 is implemented by a computer, workstation, server, or another image processor. A machine (e.g., computer, processor, server, workstation, or distributed network thereof) machine trains for use by a medical imaging system. A memory is used to store the machine-learned model and training data. The image processor (machine) trains the model, which is defined through user interaction with the image processor.

Additional, different, or fewer acts may be performed. For example, act 104 is not performed. As another example, only one, two, or three, in any combination, of acts 112-118 are performed. Acts for acquiring training data may be included. An act for distributed training may be included.

The acts are performed in the order shown (numerical or top-to-bottom) or a different order.

In act 100, a machine learning model is defined. The model is defined to receive multiple image data sets as input and to output a deformation field relating (registering or aligning) the input image data sets. In alternatives, the model is defined to output a deformed image data set based on deformation (i.e., spatially transformed for alignment).

The deformation field non-rigidly relates the spatial locations of one imaging data set to the spatial locations of the other imaging data set. The deformation field may be represented by vectors by location (e.g., pixel or voxel) for the direction and magnitude for change in spatial location. Other representations may be used, such as diffeomorphic representation provided by a velocity field, v, and a diffeomorphic deformation field, 0.

The user inputs an architecture of the model to be used in machine training. Different building blocks, such as neural network layers, activation functions, nodes, and/or other groupings, are linked together. Learnable parameters may be defined, including limits on the parameters. Fixed parameters or set values may be included or not in the definition of the architecture. Connections and weights may be defined. A default or pre-programmed model may be selected and used or altered.

The model is a neural network, such as a convolutional neural network or a fully connected neural network. The model may be a U-net, encoder-decoder, encoder-decoder with layers for a bottleneck, transformer, variational autoencoder, conditional variational autoencoder, or another image-to-image network. Support vector machines or other machine learning models may be used.

The model is defined in act 100. Acts 102 and 104 provide parts of the definition that may further assist in machine learning to generate the deformation field or deformed data. In act 102, the input and output channels are defined. The imaging data may be 3D data, such as representing a volume of the patient. Imaging data may be scan data or data to be processed into a final image or may be the end image itself. Rather than 3D, the inputs and output may be defined for 2D registration.

Unlike most tasks, DL-based 3D deformable registration requires a large hardware memory space to store inputs, outputs, and intermediate features, leaving relatively smaller memory space for model parameters. However, due to complex and a wide variety of organ size, shape, and their interaction, DL models should have a large number (e.g., 1000M, or more) of parameters (‘their innate memory’) to learn these variations and complexities. Additionally, to accurately align structures in images, the models should understand both global and local structures or features in images. In other words, unlike other tasks, hardware memory limitations adversely impact DL-based deformable registration training and performance. The number of model parameters that can be stored in the remaining hardware memory while training relative to the size of the solution spaces (i.e., all possible patient anatomy size/shape, organ size/shape, different tissue properties, motion magnitude/direction, changes and discontinuity in motions, and/or complex organ interactions) may be insufficient. As such, network architecture design is unforgiving compared to other tasks.

In act 104, the machine learning model is defined as a multi-resolution or multi-scale model. The machine learning model is a multi-resolution model with different networks for different resolutions (e.g., one network or sub-part for each resolution). Different parts of the model are provided for operating on the image data and outputting deformation at different scales. For example, the input data and resulting output deformation for each scale are downsampled or at a different resolution. Each part or network of the model operates for the different resolution.

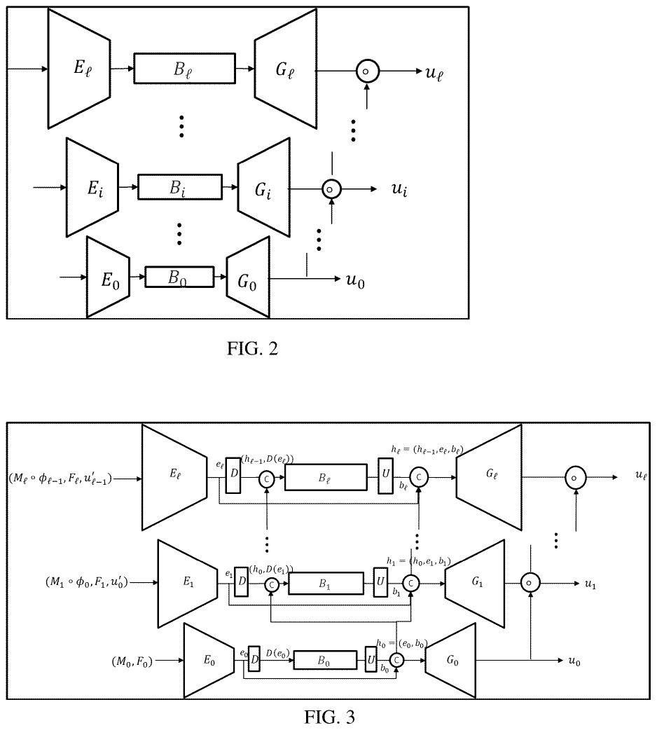

FIG. 2 shows an example network architecture. The machine learning model includes a network with separate networks (1 to l) for separate or different resolutions. Each network of the overall network includes an encoder E, bottleneck B, and decoder or generator G for multiresolution deformable registration. The bottleneck B allows the network and therefore decoders G to learn and understand the global and local image structures/features, i.e., enabling large receptive fields covering image volumes.

Registration at a single image resolution can be done by removing other registration levels, i.e., removing level 1 to e in FIG. 2. This architecture allows decoders G to see the entire image volumes (i.e., receptive fields cover image volumes). The encoders E, bottlenecks B, and decoders G are each convolutional neural network (CNN), multiple perceptorn (MLP), transformers, or other neural networks. Although the networks can incorporate transformers, the memory requirement could pose issues. For CNN, incorporation of a residual network, squeeze-and-excitation network, convolutional block attention module, dense block, and/or other specially designed blocks/modules to promote varying receptive fields and multiple paths of gradient flows may further boost network performance and allow parameters to learn key image features/structures.

In one approach, skip connections from the encoder E to the decoder G are not used but may be. Although skip connection could benefit single modality registration, skip connections could adversely affect multimodality registration by preventing the network from disregarding image intensity and only focusing on image structures.

Also, regardless of network modules (e.g., CNN, MLP, transformers), for multiresolution, it is beneficial to allow gradients to flow between levels. The architecture for 3D deformable registration allows the decoders G to see and learn both local and global structures/features (i.e., decoder's G receptive field includes the entire image volumes). Also, the architecture allows parameters to store key/necessary information and allows gradients to flow through all or multiple layers.

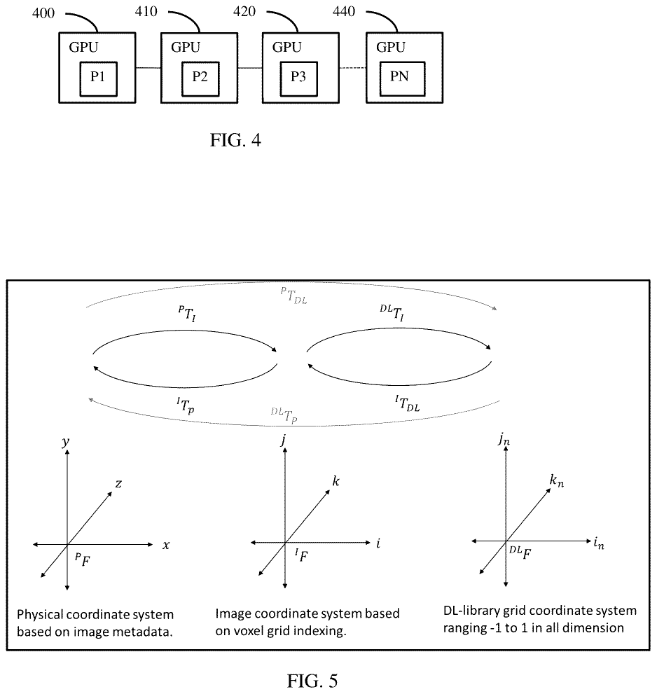

FIG. 3 shows an example of one specific network defined based on FIG. 2 where the gradients can flow from finer resolutions to coarse resolutions. The arrows represent the flow of the features. The gradients used in machine training or learning flow in the opposite direction. The network for the finest resolution is shown at l, and the network for the coarsest resolution is shown at 0. The connections between resolutions and corresponding gradient flow used in training may connect the networks for the different resolutions at any location. For example, FIG. 4 shows the connections at the input, bottleneck, and output paths, promoting stable and robust training and allowing all network parameters to learn and capture crucial information. The input connection is represented by the deformation μ from the other resolution (e.g., adjacent coarser resolution) being included in the input. The bottleneck connection provides bottleneck input e and output b (together h) from one resolution to the next resolution. The values for the features form the bottleneck are concatenated c with the values for features of that resolution network. The values of the output deformation μ from the coarser resolution are composited o with the deformation μ for the given resolution. Other connections may be used. Connection to multiple scales for a given scale may be used.

In addition to appropriate receptive fields of decoders G due to bottleneck layers B, the network promotes the understanding of global and local features via features passing between multiresolution levels, i.e., bottleneck bᵢ₋₁ and encoder features eᵢ₋₁ as well as the estimated displacement field u′ᵢ₋₁ from the previous multiresolution level i−1 to the current level i. This allows the multiresolution levels or networks to learn simultaneously with more emphasis on coarser levels since, besides gradients computed from the losses at the coarse levels, the gradients from the finer levels flow to the coarser levels, thus allowing large structures to align first but also slowly correcting misalignment of detailed structures.

In act 110, the image processor or processors train the defined machine learning model. For example, deep learning for a network as defined in act 104 is performed. Machine learning occurs, using training data to optimize the values of learnable parameters defined in the network. The optimization passes gradients to alter the values as different combinations are tested to provide the output that best matches a criterion or criteria given the inputs over a range of possible inputs (i.e., over the range of samples in the training data).

The machine training uses training data. Tens of thousands of samples are obtained, such as from experimental scans, scans of phantoms, simulations, computerized health records, a publicly available database, and/or another source. Patient records providing sample input images are provided. Imaging data from different patients may be used to form a sample input (e.g., CT from patient 1, and ultrasound from patient 2), allowing a fewer number of patient records to provide a greater number of sample inputs. Each sample includes two or more imaging data sets to be registered together, such as from the same or different modalities. For example, CT, ultrasound, MR, positron emission tomography (PET), photographic, single photon emission computed tomography (SPECT), or other types of imaging data may be provided.

The samples may represent a range of situations, such as different combinations of modalities and/or different body parts. In one implementation, the samples are for the same combination of modalities but a range of different body parts. The machine learning model is to be trained as a foundational model that can be refined for particular uses, such as refining (retraining or continued training) using sample inputs for a specific body part. In other implementations, the model is trained for a given body part and/or combination of modalities.

The machine training uses the samples to learn values of learnable parameters of the defined model. Using optimization, such as Adam or gradient descent, the values are determined by iteratively varying the values to find a combination that provides output optimized to an objective function given the range of inputs in the training data. In one embodiment, deep learning of a defined neural network is performed. Other types of machine learning may be used.

Using an objective function, unsupervised learning is provided. Rather than requiring ground truth for a large (e.g., 10k or more) set of samples, an objective function is used to measure the accuracy of deformation. In other embodiments, supervised learning (e.g., using ground truth for loss) or semi-supervised learning may be used.

For unsupervised learning, the optimization of the machine learning model uses the objective function. The objective function includes one or more factors. The factors may be similarity measurements, constraints, limits, penalties (costs), and/or rewards. For example, the factors in the objective function include an image background contribution penalty and/or a level of consistency between different resolutions constraint. The constraints or limits may be used as a penalty or cost rather than a strictly enforced boundary. Alternatively, constraints or limits are used to set a result of the objective function to a maximum or minimum so that the values resulting in violation are not used.

The objective function is to be minimized or maximized in the optimization. The factor or combination of factors forming the objective function are used as a measurement of the success of the current set of values of learnable parameters to output the proper deformation field given the input samples. The deformation field is applied, deforming one image data set to the other. The measurements are applied in a comparison of the deformed image to the base image. The measurements may additionally or alternatively be applied to the deformation field. The optimization alters the values of the learnable parameters to learn which values to use for the various parameters. The alteration is based on the value of the objective function to minimize or maximize the objective function measurement(s). The alteration explores the space of possible deformations as outputs to identify the values of the learnable parameters.

The space of deformation is large. The space is formed from all possible patient anatomy sizes and/or shapes, organ sizes and/or shapes, tissue properties, motion variations, and/or discontinuity/smooth/topology preserving. The variables of the space are continuous, so the output space for the machine learning model is very large (e.g., requiring greater than 500,000,000 model parameters to capture). The computing power and resources for training are limited. Thus, the model should have a sufficient but not-large number of parameters, such as a 10k parameters per output dimension, based on the available memory and computing resources. Although the model is not able to learn the entire solution space, the model can learn from a subspace of the solution space. The subspace of the solution space is limited or reduced. A proper subspace may be defined using deformation priors and/or constraints. The objective function may include factors to limit the solution space. Various losses, penalties, constraints, or other factors are used to form an objective function to train the machine learning model for multimodality or other deformable registration in the subspace. The extent to which the model can recover motions on a variety of imaging modalities, body parts, and motion types depends on the size of the network (‘its innate memory’).

Acts 112-116 correspond to some of the factors used in the optimization. The factors are included in the objective function for unsupervised machine training of act 110. The machine optimizes the machine learning model using the objective function, where the objective function includes one or more factors.

One or more of the factors measure similarity between the deformed image data set and the other or base image data set (i.e., between one of the input image data sets and the other of the input image data sets deformed to the one). Most widely used image similarity metrics for DL-based registration approaches are mean squared error (MSE) and (local) normalize cross correlation NCC. The former assumes the images to be registered having the same intensity distributions, and the latter assumes a linear intensity relation. Thus, both have weaknesses for multimodality registration. Dissimilarity between modality independent neighborhood descriptors (MINDs) may be used in DL for multimodality registration, but its calculation induces a large memory footprint, which, in turn, limits the model size and image resolution, hindering model ability to learn. MSE, NCC, and/or MINDs may be used for similarity measurement factors in the objective function.

In act 112, one or the only similarity factor in the objective function is a similarity measured with gradient information (GI). GI is less computationally expensive than MIND and has less weakness than MSE or NCC. The gradient information is provided as:

$$GI = {\sum }_{ℓ} \frac{1}{\vert {Ω}_{ℓ} \vert} {\sum }_{x ∈ {Ω}_{ℓ}} \frac{cos θ + 1}{2} min ( {‖ {∇}_{{ϕ}_{ℓ} ( x )} ( {M}_{ℓ} ∘ {ϕ}_{ℓ} ( x ) ) ‖}_{{2}^{′}} {‖ {∇}_{x} {F}_{ℓ} ‖}_{2} ) , \qquad ( 1 )$$

for modalities, whose intensity distributions are linearly related such as CT and cone beam CT (CBCT). For modalities whose intensities are not linearly related or less so, the gradient information may be provided as:

$$GI = {\sum }_{ℓ} \frac{1}{\vert {Ω}_{ℓ} \vert} {\sum }_{x ∈ {Ω}_{ℓ}} \frac{cos 2 θ + 1}{2} min ( {‖ {∇}_{{ϕ}_{ℓ} ( x )} ( {M}_{ℓ} ∘ {ϕ}_{ℓ} ( x ) ) ‖}_{{2}^{′}} {‖ {∇}_{x} {F}_{ℓ} ( x ) ‖}_{2} ) , \qquad ( 2 )$$

where cos θ=[ [symbol] (x))·∇ₓ [symbol] (x)]/[∥ [symbol] φₗ(x))∥₂∥∇ₓ [symbol] (x)∥₂], ∇ₓ denote the derivative with respect to x, [symbol] and [symbol] are a moving image (one image data set to be deformed) and a fixed image (another data set to be the reference) at multiresolution level [symbol] , and φₗ is a deformation at multiresolution level [symbol] . Other measures of gradient information may be used.

In act 114, a level consistency weighting for common direction is used as a factor. Where the network is a multi-scale architecture, the direction for each given location (e.g., voxel) for each resolution are rewarded where the same, discouraging different directions for different resolutions. This constraint results in the optimization seeking values that result in the direction being common across resolutions. The gradients passing between the different networks of the corresponding resolutions or scales during optimization learns to avoid opposite indications. This multiresolution level consistency constraint is a factor to reward or penalize as appropriate.

As described previously, the main issue preventing DL-based deformable registration to perform well in unseen data is the limited number of parameters per output dimension. The network may be designed so the inputs and outputs from each multiresolution level are related. As shown in FIG. 3, the architecture does not constrain the displacements at the same voxel location from different levels to be in the similar direction (i.e., consistence). Without any constraint in the objective function, the number of parameters per output dimension is |Θ|/(dΣᵢ|Ωᵢ|) where Θ denotes model parameters, d represents the spatial dimension, and Ωᵢ is the number of [symbol] image voxels at level i for i∈{1,2,3, . . . , [symbol] }. However, by constraining the displacements at the same voxel but different resolution levels to be consistent, the number of parameters per output dimension increases to |Θ|/(d| [symbol] |) (i.e., the network has more ‘innate memory’ to learn the same subspace of the solution space). The constraint enforces the direction of displacements at the same voxel to be similar across all levels and may be represented as:

$$LC = - {\sum }_{j = i + 1}^{ℓ} {\sum }_{i = 1}^{ℓ - 1} {\sum }_{x ∈ {Ω}_{i}} \frac{{u}_{i} ( x ) \cdot {u}_{j} ( x )}{{‖ {u}_{i} ( x ) ‖}_{2} {‖ {u}_{j} ( x ) ‖}_{2}} , \qquad ( 3 )$$

where uₖ(x) denotes a displacement at x at level k. A negative is used to negate the constraint since the optimization assumes minimization in one example. Other measurements of the consistency or constraint of consistency with respect to direction and/or magnitude across scales may be used.

In act 116, an image background contribution penalty is used as a factor. The background penalty weights air representation in the imaging data sets to zero displacements. The air is removed from the output space, so deformations for air are not calculated or contribute less to the objective function measurement. The background is the part of the image data sets not representing the patient. Segmentation may be used to define the background as parts of the patient, such as bone which may not move or deform. Other than zero displacement may be used, such as using a reduced or restricted displacement for the background.

In one implementation, the solution space is reduced by selecting the subspace with zero displacements outside of foreground areas in the image data sets. The image data sets may be segmented to distinguish or label the foreground from the background. This background penalty may be represented as:

$$BP = {\sum }_{ℓ} \frac{1}{\vert {Ω}_{ℓ} \vert} {\sum }_{x ∈ {Ω}_{ℓ}} {‖ {1}_{F} ( x ) {u}_{ℓ} ( x ) ‖}_{1} , \qquad ( 4 )$$

where 1_{F} is the indicator function of foreground areas (e.g., defined based on body segmentation). Other functions may be used for the background penalty.

Other factors may be provided. For example, a smoothness penalty is used for one factor. Since tissues or structures tend to move coherently, a smoothness constraint may be used model this locally coherent motion. Various smoothness constraints can be imposed. For example, diffusive regularization:

$$DR = {\sum }_{ℓ} {\sum }_{x ∈ {Ω}_{ℓ}} {‖ {\partial }_{x} {u}_{ℓ} ( x ) ‖}_{2}^{2} + {‖ {\partial }_{y} {u}_{ℓ} ( x ) ‖}_{2}^{2} + {‖ {\partial }_{z} {u}_{ℓ} ( x ) ‖}_{2}^{2} , \qquad ( 5 )$$

or bending energy penalty:

$$BE = {\sum }_{ℓ} {\sum }_{x ∈ {Ω}_{ℓ}} {‖ {H}_{x} {ϕ}_{ℓ} ( x ) ‖}_{F} , \qquad ( 6 )$$

where Hₓ denotes the Hessian and ∥⋅∥_{F} denotes the Frobenius norm, may be used. Other smoothness constraints may be used.

As another example, an inverse consistency factor is used. Unless a patient underwent surgery (e.g., tissue/implant insertion/remove), tearing and folding of tissue should not occur. To prevent such violation of topology preservation, one or more of various constraints may promote bijectivity, such as a Jacobian determinant given by:

$$JD = {\sum }_{ℓ} \frac{1}{\vert {Ω}_{ℓ} \vert} {\sum }_{x ∈ {Ω}_{ℓ}} max ( 0 , - det ( {∇}_{x} {ϕ}_{ℓ} ( x ) ) ) , \qquad ( 7 )$$

to minimize negative Jacobian determinants of deformation. In cases where mappings are computed in both directions for each multiresolution level [symbol] , i.e. [symbol] maps a moving image to a fixed image and

$${ϕ}_{ℓ}^{′}$$

maps the fixed image to the moving image, inverse consistency may be imposed as:

$$IC = {\sum }_{ℓ} \{ \frac{1}{\vert {}^{F} {Ω}_{ℓ} \vert} {\sum }_{x ∈ {}^{F} {Ω}_{ℓ}} {‖ {ϕ}_{ℓ}^{′} ∘ {ϕ}_{ℓ} ( x ) - x ‖}_{2}^{2} + \frac{1}{\vert {}^{M} {Ω}_{ℓ} \vert} {\sum }_{y ∈ {}^{M} {Ω}_{ℓ}} {‖ {ϕ}_{ℓ} ∘ {ϕ}_{ℓ}^{′} ( y ) - y ‖}_{2}^{2} \} , \qquad ( 8 )$$

where ^{M} [symbol] and ^{F} [symbol] denote the moving and fixed grid, respectively, or a weakly form defined in terms of the spatial Jacobians, given as:

$$JIC = {\sum }_{ℓ} \{ \frac{1}{\vert {}^{F} {Ω}_{ℓ} \vert} {\sum }_{x ∈ {}^{F} {Ω}_{ℓ}} {‖ {∇}_{{ϕ}_{ℓ} ( x )} ( {ϕ}_{ℓ}^{′} ∘ {ϕ}_{ℓ} ( x ) ) {∇}_{x} ϕ ( x ) - I ‖}_{F} + \frac{1}{\vert {}^{M} {Ω}_{ℓ} \vert} {\sum }_{y ∈ {}^{M} {Ω}_{ℓ}} {‖ {∇}_{{ϕ}_{ℓ}^{′} ( y )} ( {ϕ}_{ℓ} ∘ {ϕ}_{ℓ}^{′} ( y ) ) {∇}_{y} {ϕ}_{ℓ}^{′} ( y ) - I ‖}_{F} \} , \qquad ( 9 )$$

where I∈ [symbol] ^{d×d} denotes the identity matrix. Other inverse consistency [symbol] functions may be used.

In cases where mappings are computed in both directions, GI in eqs. (1-2), LC in eq. (3), BP in eq. (4), DR in eq. (5), BE in eq. (6), and JD in eq. (7) for the inverse direction can be derived and imposed, similarly.

Other factors, such as differentiable DICE, may be used or included in the objective function to further constrain the solution space and improve training stability and model performance at inference. Any objective function using the included factors may be provided. For example, one or more factors act as limits or boundaries where one or more factors contribute to the measurement. In one example, the total objective function is the weighted sum of all the factors (e.g., loss terms and penalty terms) as:

$${arg min}_{Φ , {Φ}^{′}} {λ}_{GI} GI + {λ}_{LC} LC + {λ}_{BP} BP + {λ}_{DR} DR + {λ}_{BE} BE + {λ}_{JD} JD + {λ}_{IC} IC + {λ}_{JIC} JIC ,$$

where Φ={ [symbol] } and

$${Φ}^{′} = \{ {ϕ}_{ℓ}^{′} \}$$

and for displacement field-based deformation,

$${ϕ}_{ℓ} = x + {\sum }_{i}^{ℓ} {u}_{i} ( x ) ,$$

and the same for

$${ϕ}_{ℓ}^{′} .$$

Any weights, λ, may be used for the factors.

In act 118, the regularization in the optimization uses spatial Jacobians with a defined normalized grid unit (NGU). The regularization may use bending energy, diffusive prior, inverse consistency (e.g., ReLU of negative of Jacobian determinates, spatial Jacobians of composition of forward and backward deformation), or another smoothness operation or function (e.g., Gaussian filter) may be used.

The spatial units (e.g., NGU) are defined with the coordinate frames of the estimated motions (deformations). This allows spatial Jacobians to be correctly computed to impose regularization, the penalty encouraging regularity and bijectivity, and/or other prior knowledge whose definition involves the spatial Jacobians of deformation or other smoothness operation.

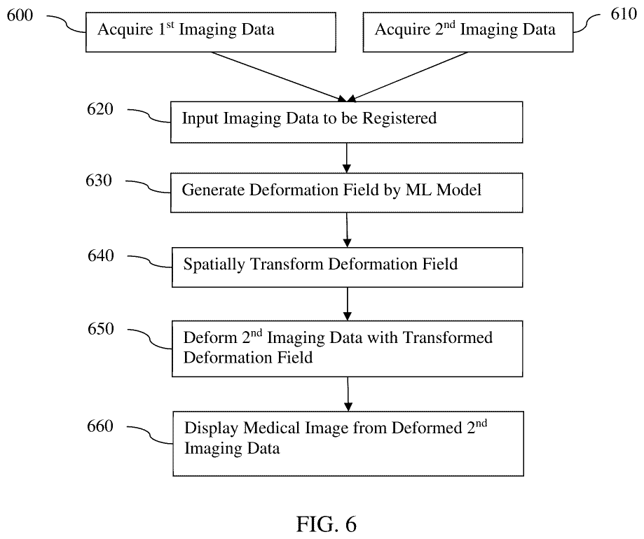

Spatial coordinate system transformation is provided. One of leading hindrances of the application of DL-based registration in clinical workflow is the mismatches and/or disregard of image spatial information as defined in image metadata. This spatial information assists in accurate fusion and analyses of images acquired from the same or multiple systems, at the same or different time points, from the same or different institution, and from the same or different patients. The spatial information may be incorporated through the chain of coordinate systems, allowing transformations to be used to move between coordinate systems. In application (inference), deformations may be transformed to the physical coordinate system as defined in the image metadata (see act 640 of FIG. 6 and the corresponding discussion below).

FIG. 5 shows coordinate system transformations between the physical image grid ^{P}F as defined by image metadata, the voxel index grid as defined by the number of voxels per dimension ^{I}F, and the DL image grid ^{DL}F as defined by DL libraries. ^{A}T_{B} is a transformation for the change of coordinate system from ^{B}F to ^{A}F. As shown in FIG. 5, the image physical coordinate system ^{P}F is similar to the definition used in Insight Toolkit (ITK) based libraries. Given image spacing S∈ [symbol] ^{d×d}, image orientation D∈ [symbol] ^{d×d} and image origin t∈ [symbol] ^{d×1}, a voxel index i may be converted to a physical point x by ^{P}T_{I}i=DSi+t=[DS t]i=x. Its inverse allows conversion in the opposite direction from x to i, ^{I}T_{P}x=(DS)⁻¹x−(DS)⁻¹t=[(DS)⁻¹−(DS)⁻¹t]x=i. Similarly, transformations ^{DL}T_{I} and ^{I}T_{DL} are defined between the voxel index coordinate system ^{I}F and the DL-libraries image grid ^{DL}F. Let sᵢ, sⱼ and sₖ denote the number of voxels in each image direction for 3D. The transformation from ^{I}F to ^{DL}F is:

$${DL}^{} {T}_{I} = [ [ \begin{matrix}2 / ( {s}_{i} - 1 ) & 0 & 0 \\ 0 & 2 / ( {s}_{j} - 1 ) & 0 \\ 0 & 0 & 2 / ( {s}_{k} - 1 )\end{matrix} ] [ \begin{matrix}- 1 \\ - 1 \\ - 1\end{matrix} ] ] = [ \begin{matrix}{}^{DL} {A}_{I} & {}^{DL} {t}_{I}\end{matrix} ]$$

and its inverse allows movement in the opposite direction:

$$\begin{matrix}{}^{I} {T}_{DL} = [ [ \begin{matrix}( {s}_{i} - 1 ) / 2 & 0 & 0 \\ 0 & ( {s}_{j} - 1 ) / 2 & 0 \\ 0 & 0 & ( {s}_{k} - 1 ) / 2\end{matrix} ] [ \begin{matrix}( {s}_{i} - 1 ) / 2 \\ ( {s}_{j} - 1 ) / 2 \\ ( {s}_{k} - 1 ) / 2\end{matrix} ] ] \\ = [ \begin{matrix}{}^{I} {A}_{DL} & {}^{I} {t}_{DL}\end{matrix} ] ,\end{matrix}$$

following the coordinate system definition defined by DL libraries, for example PyTorch. As shown in FIG. 5, given these four transformations, the frame of reference may move between coordinate systems as well as changing from the DL-grid coordinates to the physical coordinates ^{P}T_{DL}=^{P}T_{I} ^{I}T_{DL} and vice versa ^{DL}T_{P}=^{DL}T_{I} ^{I}T_{P}.

The coordinate transformations allow estimation of deformation in the DL-grid coordinate system and return the estimated deformation in the physical coordinate system. The physical coordinate system is the coordinate system assumed and used by other existing applications and systems in clinics for image registration. By transforming the deformation from the DL coordinate system to the physical coordinate system, more accurate deformations for use in registration or alignment are provided. Consider estimated deformations of the form i′ₙ=ψₙ(iₙ)=iₙ+uₙ(iₙ)∈^{DL}F, the corresponding deformation in the physical coordinate system is given by y=ψ(x)=x+u(x)∈^{P}F, where x=^{P}T_{DL}iₙ and u=DS^{I}A_{DL}uₙ. In other words, y=^{P}T_{DL}iₙ+DS^{I}A_{DL}uₙ(^{P}T_{DL}iₙ).

Similarly, in training, the deformations may be transformed for deforming the image data set of the sample input to calculate similarity for the objective function. Using this clear convention of coordinate systems and their transformations, the unit of deformations is known. Displacements estimated during training (optimization) are defined in ^{DL}F in a normalized grid unit (NGU). Likewise, the voxel spacing in ^{DL}F is known, i.e., [2/(sₓ−1)2/(s_{y}−1) 2/(s_{z}−1)] NGU/voxel for 3D.

The unit of deformations may also be used in regularization of optimization in act 118. Finite differences may be used to estimate the spatial Jacobians of the deformation and their determinants which, in this case, will be in voxels and voxel³ correctly representing relative change in local volume due to scaling. For example, the central difference for spatial Jacobian can be computed as:

$${∇}_{x} u = [ \begin{matrix}{\partial }_{x} {u}_{x} & {\partial }_{y} {u}_{x} & {\partial }_{z} {u}_{x} \\ {\partial }_{x} {u}_{y} & {\partial }_{y} {u}_{y} & {\partial }_{z} {u}_{y} \\ {\partial }_{x} {u}_{y} & {\partial }_{y} {u}_{z} & {\partial }_{z} {u}_{z}\end{matrix} ] \approx [ \begin{matrix}\frac{[ {u}_{x} ( {i}_{n} + {ϵ}_{x} ) - {u}_{x} ( {i}_{n} - {ϵ}_{x} ) ]}{2 {ϵ}_{x}} & \frac{[ {u}_{x} ( {i}_{n} + {ϵ}_{y} ) - {u}_{x} ( {i}_{n} - {ϵ}_{y} ) ]}{2 {ϵ}_{y}} & \frac{[ {u}_{x} ( {i}_{n} + {ϵ}_{z} ) - {u}_{x} ( {i}_{n} - {ϵ}_{z} ) ]}{2 {ϵ}_{z}} \\ \frac{[ {u}_{y} ( {i}_{n} + {ϵ}_{x} ) - {u}_{y} ( {i}_{n} - {ϵ}_{x} ) ]}{2 {ϵ}_{x}} & \frac{[ {u}_{y} ( {i}_{n} + {ϵ}_{y} ) - {u}_{y} ( {i}_{n} - {ϵ}_{y} ) ]}{2 {ϵ}_{y}} & \frac{[ {u}_{y} ( {i}_{n} + {ϵ}_{z} ) - {u}_{y} ( {i}_{n} - {ϵ}_{z} ) ]}{2 {ϵ}_{z}} \\ \frac{[ {u}_{z} ( {i}_{n} + {ϵ}_{x} ) - {u}_{z} ( {i}_{n} - {ϵ}_{x} ) ]}{2 {ϵ}_{x}} & \frac{[ {u}_{z} ( {i}_{n} + {ϵ}_{y} ) - {u}_{z} ( {i}_{n} - {ϵ}_{y} ) ]}{2 {ϵ}_{y}} & \frac{[ {u}_{z} ( {i}_{n} + {ϵ}_{z} ) - {u}_{z} ( {i}_{n} - {ϵ}_{z} ) ]}{2 {ϵ}_{z}}\end{matrix} ]$$

where ϵₖ=2/(sₖ−1). Since uₖ is in NGU and ϵₖ is NGU/voxel, ∇ₓu is in voxel and det(∇ₓu) is in voxel³, correctly measuring relative change in volume size. Unlike this approach, existing DL approaches define spatial Jacobians of the deformations used in regularization/penalty terms with improper units due to the unknown unit of estimated deformations and the coordinate systems in which the estimated deformations reside. These regularizers and/or penalties, instead of properly imposing smoothness or their intended prior knowledge, induce irregularity of the estimated deformation and tearing and folding of tissues. By integrating this coordinate transformation chain, the DL approach returns deformations that can be directly integrated into current clinical workflows and current image analysis tasks.

To take advantage of available computing resources and memory, the optimization of act 110 may use training distributed across different computers. For example, and as shown in FIG. 4, the learnable parameters P1-N are distributed across different computers (e.g., graphics processing units 400, 410, 420, . . . 440). Alternatively, distribution of training data is used. In other implementations, a single workstation, server, or computer trains (i.e., no distribution or only distribution amongst locally available cores or processors).

In addition to network design, the training scheme may facilitate the memory limitation, allowing the model to have as many parameters as possible. A single computing unit (e.g., a single GPU 400) may not be sufficient for some networks. Likewise, distributed training based purely on data partition and/or parallel operation also includes duplicate copies of the whole model, thus limiting the number of parameters. Instead, both model and data are partitioned across computing units. The example of FIG. 4 is a simple example of partitioning model parameters P across computing units (GPU) using a fully shared data parallel scheme. Each GPU 400-440 is responsible for one or more parameters and communicates with the other GPUs 400-440 to pass feature values during training. Partitioning both model parameters and data allow the model to have the largest number of model parameters for the available computing resources. In one example, more than 48 GPUs (each 80 GB) are used to train a foundation model with greater than 50k image pairs. This may allow learning all possible deformations.

Each parameter or parameter group is stored and trained on a separate GPU 400-440. Padding may be appended to the model parameters. Various sharding schemes may be used. Parameters may be provided for model weights and bias.

In act 120, the image processor stores the machine-trained model as optimized. After training, the defined architecture, values of the learnable parameters, and values of set parameters are stored. Any format may be used.

The machine-trained model is stored in a memory, such as a memory local to the image processor. Additionally, or alternatively, the machine-trained model is transmitted or delivered to one or more (e.g., multiple) memories, such as at different healthcare facilities and/or medical scanners.

The values of the stored or trained machine-learned model are fixed. The values of the learnable parameters are set for application (inference) for one or more patients. The values do not change during application for a patient. The machine-learned model may be retrained or updated using additional training data. For example, the trained model is a foundational model, which is then refined with further training using training data for a particular body part and/or combination of modalities.

FIG. 6 shows one embodiment of a method for registration by a medical imaging system. A machine-learned model is applied to infer deformation for spatially registers two or more input images. The model as trained according to the method of FIG. 1 or other machine training is applied for a specific patient. For example, a processor uses the previously trained network of FIG. 2 or FIG. 3 to generate a deformation field. The use of GI, level consistency, background penalty, and/or multi-scale with connections between resolutions in the training results in a machine-learned model better able to generate an accurate deformation field in application or testing. A transform may be applied to the deformation field prior to application to the image data, providing accurate deformation in the physical coordinate system of the image data.

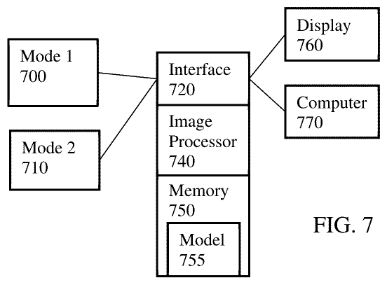

The method of FIG. 6 is implemented by a medical imaging system. The medical imaging system may be a computer, server, workstation, medical scanner, and/or other processing device. For example, the medical imaging system of FIG. 7 is used. Medical scanners perform acts 600 and 610. A processor performs acts 620-640. The same or a different processor performs act 650. A display, controlled by a processor, performs act 660. Other devices or components may be used instead or in addition to the imaging systems and/or processors.

Additional, different, or fewer acts may be performed. For example, acts 600 and 610 are not provided, such as where the imaging data is already provided. As another example, act 640 is not performed. In another example, acts 650 and/or 660 are not performed, such as where an existing clinical application is to later use the deformation field. One application or program may provide the deformation by performing acts 620-630, and optionally act 640, and another existing clinical application or program for registration or viewing images may perform acts 650 and/or 660. Alternatively, the same application or program performs acts 620-660.

The acts are performed in the order shown (numerical or top-to-bottom) or a different order. Acts 600 and 610 may be performed in any order, such as initially acquiring pre-operative data and later acquiring intra- or post-operative data.

In acts 600 and 610, an image processor acquires different sets of imaging data. The acquisition may be from scanning a patient. The acquisition may be from memory or transfer over a computer network, such as loading one or both sets of imaging data from a picture archival and communications system (PACS).

The two sets of imaging data are from the same modality, such as using different or the same system to scan the patient at different times. Alternatively, the two sets of imaging data from different modalities, such as using two different types (modalities) of scanners (i.e., different imaging physics and/or sensor arrangements) to scan the patient at the same or different times. The timing may be different, such as one set of imaging data being from a pre-operative scan, and the other set of imaging data being from an intra-operative scan.

The imaging data sets are volumes, such as reconstructed or formed voxel representations of the patient. Both sets of imaging data represent a same or overlapping region of the patient, such as an organ, organs, and/or anatomy of interest. Due to patient motion, changes in the patient over time, differences in the scanners, or other difference, particular locations in one volume may be represented at different locations in the other volume. The imaging data sets are or are not aligned.

The imaging data may be linked with metadata. For example, the imaging data sets are in a digital imaging and communications in medicine (DICOM) format with metadata in the header. As another example, the metadata is encoded in the imaging data or stored at a referenced memory location. The metadata provides spatial information for the imaging data, such as the spacing, orientation, and origin for the samples of the represented volume of the patient. This, additional, or different information may provide an indication of the physical coordinate system with reference to the scanner and/or the patient.

In act 620, the image processor inputs the imaging data sets to a machine-learned model. The image processor loads the machine-learned model and provides the imaging data sets to the input channels of the machine-learned model.

In act 630, the machine-learned model, implemented by the image processor, generates a deformation field for deforming one imaging data to the other imaging data. The image processor applies the machine-learned model to estimate deformations to register the input image data.

In response to the input, the machine-learned model generates an output. The output is a deformation field representing a non-rigid spatial transform between the different sets of image data. Alternatively, the machine-learned model outputs the deformed or spatially transformed image data of one of the sets.

The output by the machine-learned model is based on the previously performed machine training. Different training data, architecture definition, type of training, objective function used in training, regularization, and/or training settings result in different values of the learnable parameters, so result in different machine-learned models. The previously performed training determines, in part, the output generated by the machine-learned model in response to input of the previously unseen imaging data for the particular patient. Any one or more of the options discussed below trained the machine-learned model now being applied.

In one option, the machine-learned model is a deep learned neural network. In one implementation, the machine-learned model is a multi-scale or multi-resolution model. Networks at different resolutions have input channels receiving the image data at different resolutions. In training, gradients connections were included to pass gradients in optimization between the different resolutions (between networks at different scales), such as shown in FIG. 3. For example, the connections are for input, the bottleneck, and output of the networks, such as shown in FIG. 3. The connections may or may not be for features used in application. Other machine-learned models may be used, such as multi-scale without the connections or with different connections (e.g., a subset and/or originating or ending within the encoder, decoder, and/or bottleneck).

In another option, the machine-learned model was trained using know spatial coordinate systems. The deformation field is output in a coordinate system of the machine-learned model, which may be different than the coordinate system of the image data. By using a normalized grid in a known coordinate system in training the model, finite differences may be used to estimate spatial Jacobians of deformations and the determinants of spatial Jacobians. The accuracy of the machine-learned model may, in part, be due to using the normalized grid and transformations relative to the physical coordinate system in training.

As some other options, the machine-learned model was trained with an objective function in an unsupervised manner. The objective function used or included a similarity metric using gradient information. Alternatively, or additionally, the objective function included a multi-resolution level consistency factor to define the output space (subspace). Alternatively, or additionally, the objective function included a background penalty factor to define the output space. Other information may have been included in the objective function.

In act 640, the image processor spatially transforms the deformation field from the coordinate system of the machine-learned model to a physical coordinate system. The physical coordinate system is known or accessed from the linked metadata. For example, the spacing, orientation, and origin define the physical coordinate system for the image data to which the deformation is to be applied. The coordinate system of the machine-learned model is known, such as from the normalized grid used in training and/or defined by the library of training data. Due to differences in the coordinate systems, the transformation between the coordinate systems converts the deformation field output from the machine-learned model into the coordinate system of the image data to which the deformation is to be applied. The transformation is applied to the deformation field. Alternatively, the machine-learned model outputs the image data as transformed in the physical coordinate system, or the transform is applied to deformed image data.

In act 650, the image processor deforms one set of image data. The deformation field as transformed to the physical coordinate system of the image data is applied to the image data. Each voxel or sample is shifted in a direction and magnitude defined by the deformation field. This non-rigid deformation aligns or registers the image data being deformed with the other or reference image data.

In act 660, a display displays a medical image of the patient from the image data as deformed. The image processor uses the image data to generate the medical image, such as rendering an image from the volume representing the patient. The image is of the patient, such as an interior region of the patient.

The image is rendered, such as three-dimensional rendered, from the voxels of the image data to a two-dimensional display image. Alternatively, multi-planar reconstruction or planar imaging uses data representing a plane or planes to generate a two-dimensional image for the two-dimensional display.

Due to the registration (deformation), the medical image may include images from both sets of image data in an aligned manner. A location in one image relates to the same location in the other image, assisting in understanding during side-by-side or even sequential viewing. One image may be overlaid on another using the registration.

FIG. 7 shows one embodiment of a system for image registration in medical imaging. The system applies the machine-learned model 755, such as described above for FIG. 6.

The system includes two or more scanners 700, 710, such as scanners of different modalities. The system includes an interface 720, image processor 740, memory 750, display 760, and/or computer 770. Additional, different, or fewer components may be provided, such as the interface 720 and/or memory 750 and the image processor 740 without the scanners 700, 710, display 760, and/or computer 770.

The scanners 700, 710 are of any medical imaging modality. CT, MR, ultrasound, PET, SPECT, x-ray, optical, and/or other imaging modalities may be used. The scanners 700, 710 scan the patient, providing imaging data representing the same or overlapping volumes in the patient.

The interface 720 is a network card, memory interface, display buffer, and/or computer communications device. The interface 720 is configured by protocol and/or the image processor 740 to receive images representing the patient, such as receiving volumes form the scanners 700, 710 and/or the memory 750. For example, images are received from different modalities. The interface 720 is configured to output the deformation field, such as outputting to the computer 770, the display 760, the memory 750, and/or the scanner(s) 700, 710. The interface 720 may instead output deformed images.

The image processor 740 is a general processor, digital signal processor, graphics processing unit, application specific integrated circuit, field programmable gate array, artificial intelligence processor, tensor processor, digital circuit, analog circuit, combinations thereof, or other now known or later developed device for estimating deformation, spatially transforming, applying a deformation, generating an image, and/or registering images. The processor 740 is a single device, a plurality of devices, or a network. For more than one device, parallel or sequential division of processing may be used. Different devices making up the processor 740 may perform different functions, such as one processor for estimating deformation and another processor for generating images using the deformation. In one embodiment, the processor 740 is a control processor or other processor of one of the scanners 700, 710. In other embodiments, the processor 740 is part of a separate workstation, server, or computer.

The image processor 740 operates pursuant to stored instructions to perform various acts described herein. The image processor 740 is configured by software, design, firmware, and/or hardware to perform any or all the acts of FIG. 6.

The image processor 740 is configured to input images to the machine-learned network 755. The machine-learned network 755 is configured by previous training to output a deformation field in response to the input. The objective function used in training (e.g., level consistency, GI similarity, and/or background penalty), the use of known coordinates in training (e.g., displacements defined in normalized grid), and/or the architecture of the network (e.g., multi-resolution with connections between resolutions) result in the machine-learned network 755 operating to output an accurate deformation field for deforming one of the input images to the other of the input images. In one implementation, the machine-learned network 755 is a multi-resolution network having been trained with gradients passing between resolutions of the multi-resolution network in optimization of the multi-resolution network.

The deformation field as output is defined in a coordinate system based on the training data. This machine-learned network coordinate system may be different than the physical coordinate system(s) of the input images. The image processor 740 is configured to spatially transform the deformation field to the physical coordinate system of one of the input images. Metadata defining the coordinate system of the input image is used to generate the transformation applied by the image processor 740.

The image processor 740 is configured to register the input images. The deformation field, such as after transformation to the physical coordinate system, is applied to one of the input images. The deformed image is registered with the other image due to the deformation. The same locations in the patient are represented in the same locations in both images as registered. The image processor 740 may be configured to generate one or more medical images using the registered images.

The images (imaging data), machine-learned network 755, features calculated by the machine-learned network 755, deformation field, transformations, metadata, medical images, and/or other data are stored in the memory 750. The data is stored in any format. The memory 750 is a buffer, cache, RAM, removable media, hard drive, magnetic, optical, database, or other now known or later developed memory. The memory 750 is a single device or group of two or more devices. The memory 750 is part of the scanner 700, 710, a computer, workstation, or database, such as a PACS memory.

The memory 750 is additionally or alternatively a non-transitory computer readable storage medium with processing instructions. The memory 750 stores data representing instructions executable by the programmed processor 740 for deformation estimation and/or image registration or by an image processor for machine training. The instructions for implementing the processes, methods and/or techniques discussed herein are provided on non-transitory computer-readable storage media or memories, such as a cache, buffer, RAM, removable media, hard drive, or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The display 760 is a CRT, LCD, plasma screen, projector, printer, or other output device for showing a medical image. Images as registered are displayed adjacently, overlaid, and/or sequentially. An image of the deformation field may be displayed. The medical image may be generated as a three-dimensional rendering, multi-planar reconstruction, or two-dimensional imaging of the patient. The medical images are displayed on the display.

The computer 770 is configured to implement a clinical application or program. In embodiments where the image processor 740 and interface 720 output a deformation field, the computer 770 may use the deformation field for registration.

Listed below are various Illustrative Embodiments. The Illustrative Embodiments summarize different combinations of aspects. Other combinations of any of the aspects with any other one or more of the aspects may be provided. Aspects from one type (e.g., method or system) may be used in another type (system or method).

Illustrative Embodiment 1. A method for deformable registration by a medical imaging system, the method comprising: inputting, by an image processor, first imaging data and second imaging data to a machine-learned model; generating, by the machine-learned model and in response to the inputting, a deformation field deforming the second imaging data to the first imaging data, the deformation field being in a coordinate system of the machine-learned model; spatially transforming the deformation field from the coordinate system of the machine-learned model to a physical coordinate system; deforming the second imaging data by the deformation field as transformed; and displaying a medical image of the patient from the second imaging data as deformed.

Illustrative Embodiment 2. The method of Illustrative Embodiment 1, wherein the second imaging data is linked with metadata for spacing, orientation, and origin, and wherein spatially transforming comprises spatially transforming with the physical coordinate system defined by the spacing, orientation, and origin where the coordinate system of the machine-learned model is different than the physical coordinate system and corresponds to a library of training data.

Illustrative Embodiment 3. The method of any of Illustrative Embodiments 1-2, wherein the first and second imaging data are from different medical imaging modalities, wherein deforming and displaying are performed by a clinical imaging application and inputting, generating, and spatially transforming are performed by separate application.

Illustrative Embodiment 4. The method of any of Illustrative Embodiments 1-3, wherein generating by the machine-learned model comprises generating where the machine-learned model was trained with the training data in a normalized grid.

Illustrative Embodiment 5. The method of Illustrative Embodiment 4, wherein the machine-learned model was trained using finite differences to estimate spatial Jacobians of deformation and the determinants of the spatial Jacobians.

Illustrative Embodiment 6. The method of any of Illustrative Embodiments 1-5, wherein generating by the machine-learned model comprises generating by the machine-learned model comprising a plurality of networks at different resolutions where the networks were trained using gradient connections between the networks at the different resolutions.

Illustrative Embodiment 7. The method of Illustrative Embodiment 6, wherein the gradient connections comprise input, bottleneck, and output connections of the networks.

Illustrative Embodiment 8. The method of any of Illustrative Embodiments 1-7, wherein generating by the machine-learned model comprises generating by the machine-learned model having been trained with an objective function in an unsupervised manner, the objective function comprising a similarity metric using gradient information.

Illustrative Embodiment 9. The method of any of Illustrative Embodiments 1-8, wherein generating by the machine-learned model comprises generating by the machine-learned model having been trained with an objective function that defined an output space, at least in part, with a multi-resolution level consistency factor.

Illustrative Embodiment 10. The method of any of Illustrative Embodiments 1-9, wherein generating by the machine-learned model comprises generating by the machine-learned model having been trained with an objective function that defined an output space, at least in part, with a background penalty.

Illustrative Embodiment 11. A method for machine training for deformable registration by a medical imaging system, the method comprising: defining a machine learning model with input of multiple image data sets and output of a deformation field relating the multiple image data sets, optimizing the machine learning model using an objective function, the objective function including a factor for image background contribution penalty and/or for level consistency between different resolutions; and storing the machine learning model as optimized.

Illustrative Embodiment 12. The method of Illustrative Embodiment 11, wherein optimizing comprises optimizing using the factor comprising the image background contribution penalty and the level consistency.

Illustrative Embodiment 13. The method of any of Illustrative Embodiments 11-12, wherein optimizing comprises optimizing using the factor comprising the image background contribution penalty weighting air representation in the image data sets to zero displacements.

Illustrative Embodiment 14. The method of any of Illustrative Embodiments 11-13, wherein defining comprises defining the machine learning model to be a multi-resolution model with different networks for different ones of the different resolutions; and wherein optimizing comprises optimizing using the factor comprising the level consistency weighting common direction at the different resolutions and with gradients passing between the different networks, the objective function including a similarity measured with gradients.

Illustrative Embodiment 15. The method of any of Illustrative Embodiments 11-14, wherein optimizing comprises optimizing with regularization using spatial Jacobians with a defined normalized grid unit.

Illustrative Embodiment 16. The method of any of Illustrative Embodiments 11-15, wherein optimizing comprises optimizing with learnable parameters of the machine learning model distributed across different computers.

Illustrative Embodiment 17. A method for machine training for deformable registration by a medical imaging system, the method comprising: defining a machine learning model with input of multiple image data sets and output of a deformation field relating the multiple image data sets; optimizing the machine learning model using an objective function, the objective function including a first factor for similarity, the similarity measured with gradient information; and storing the machine learning model as optimized.

Illustrative Embodiment 18. The method of Illustrative Embodiment 17 wherein optimizing comprises optimizing with the objective function including a second factor, the second factor comprising an image background contribution penalty and/or a level consistency between different resolutions.

Illustrative Embodiment 19. The method of any of Illustrative Embodiments 17-18, wherein optimizing comprises optimizing with regularization using spatial Jacobians with a defined normalized grid unit.

Illustrative Embodiment 20. The method of any of Illustrative Embodiments 17-19, wherein defining comprises defining the machine learning model to be a multi-resolution model with different networks for different ones of different resolutions; and wherein optimizing comprises optimizing with gradients passing between the different networks.

Illustrative Embodiment 21. A system for image registration in medical imaging, the system comprising: an interface configured to receive first and second images representing a patient, the first and second images from different imaging modalities; a memory configured to store a machine-learned network, the machine-learned network comprises a multi-resolution network having been trained with gradients passing between resolutions of the multi-resolution network in optimization of the multi-resolution network; and a processor configured to input the first and second images to the machine-learned network, the machine-learned network configured to output a deformation field in response to the input; wherein the interface is configured to output the deformation field.

Illustrative Embodiment 22. The system of Illustrative Embodiment 21 wherein the deformation field as output is defined in a first coordinate system based on the training data, and wherein the processor is configured to spatially transform the deformation field to a second coordinate system based on metadata of the first image.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for deformable registration by a medical imaging system, the method comprising:
inputting, by an image processor, first imaging data and second imaging data to a machine-learned model;
generating, by the machine-learned model and in response to the inputting, a deformation field deforming the second imaging data to the first imaging data, the deformation field being in a coordinate system of the machine-learned model;
spatially transforming, by the image processor, the deformation field from the coordinate system of the machine-learned model to a physical coordinate system;
deforming the second imaging data by the deformation field as transformed; and
displaying a medical image of the patient from the second imaging data as deformed.

2. The method of claim 1, wherein the second imaging data is linked with metadata for spacing, orientation, and origin, and wherein spatially transforming comprises spatially transforming with the physical coordinate system defined by the spacing, orientation, and origin where the coordinate system of the machine-learned model is different than the physical coordinate system and corresponds to a library of training data.

3. The method of claim 1, wherein the first and second imaging data are from different medical imaging modalities, wherein deforming and displaying are performed by a clinical imaging application and inputting, generating, and spatially transforming are performed by separate application.

4. The method of claim 1, wherein generating by the machine-learned model comprises generating where the machine-learned model was trained with the training data in a normalized grid.

5. The method of claim 4, wherein the machine-learned model was trained using finite differences to estimate spatial Jacobians of deformation and the determinants of the spatial Jacobians.

6. The method of claim 1, wherein generating by the machine-learned model comprises generating by the machine-learned model comprising a plurality of networks at different resolutions where the networks were trained using gradient connections between the networks at the different resolutions.

7. The method of claim 6, wherein the gradient connections comprise input, bottleneck, and output connections of the networks.

8. The method of claim 1, wherein generating by the machine-learned model comprises generating by the machine-learned model having been trained with an objective function in an unsupervised manner, the objective function comprising a similarity metric using gradient information.

9. The method of claim 1, wherein generating by the machine-learned model comprises generating by the machine-learned model having been trained with an objective function that defined an output space, at least in part, with a multi-resolution level consistency factor.

10. The method of claim 1, wherein generating by the machine-learned model comprises generating by the machine-learned model having been trained with an objective function that defined an output space, at least in part, with a background penalty.

11. A method for machine training for deformable registration by a medical imaging system, the method comprising:
defining a machine learning model with input of multiple image data sets and output of a deformation field relating the multiple image data sets;
optimizing, by a processor, the machine learning model using an objective function, the objective function including a factor for image background contribution penalty and/or for level consistency between different resolutions; and
storing the machine learning model as optimized.

12. The method of claim 11, wherein optimizing comprises optimizing using the factor comprising the image background contribution penalty and the level consistency.

13. The method of claim 11, wherein optimizing comprises optimizing using the factor comprising the image background contribution penalty weighting air representation in the image data sets to zero displacements.

14. The method of claim 11, wherein defining comprises defining the machine learning model to be a multi-resolution model with different networks for different ones of the different resolutions; and
wherein optimizing comprises optimizing using the factor comprising the level consistency weighting common direction at the different resolutions and with gradients passing between the different networks, the objective function including a similarity measured with gradients.

15. The method of claim 11, wherein optimizing comprises optimizing with regularization using spatial Jacobians with a defined normalized grid unit.

16. The method of claim 11, wherein optimizing comprises optimizing with learnable parameters of the machine learning model distributed across different computers.

17. A method for machine training for deformable registration by a medical imaging system, the method comprising:
defining a machine learning model with input of multiple image data sets and output of a deformation field relating the multiple image data sets;
optimizing, by a processor, the machine learning model using an objective function, the objective function including a first factor for similarity, the similarity measured with gradient information; and
storing the machine learning model as optimized.

18. The method of claim 17 wherein optimizing comprises optimizing with the objective function including a second factor, the second factor comprising an image background contribution penalty and/or a level consistency between different resolutions.

19. The method of claim 17, wherein optimizing comprises optimizing with regularization using spatial Jacobians with a defined normalized grid unit.

20. The method of claim 17, wherein defining comprises defining the machine learning model to be a multi-resolution model with different networks for different ones of different resolutions; and
wherein optimizing comprises optimizing with gradients passing between the different networks.

21. A system for image registration in medical imaging, the system comprising:
an interface configured to receive first and second images representing a patient, the first and second images from different imaging modalities;
a memory configured to store a machine-learned network, the machine-learned network comprises a multi-resolution network having been trained with gradients passing between resolutions of the multi-resolution network in optimization of the multi-resolution network; and
a processor configured to input the first and second images to the machine-learned network, the machine-learned network configured to output a deformation field in response to the input;
wherein the interface is configured to output the deformation field.

22. The system of claim 21 wherein the deformation field as output is defined in a first coordinate system based on the training data, and wherein the processor is configured to spatially transform the deformation field to a second coordinate system based on metadata of the first image.
